(19) United States
(12) Patent Application Publication
(10) Pub. No.: US 2024/0091447 A1
(43) Pub. Date: Mar. 21, 2024

(54) INJECTION DEVICE

(71) Applicant: Owen Mumford Limited, Woodstock, Oxfordshire (GB)

(72) Inventors: Toby Ken COWE, Woodstock, Oxfordshire (GB); Timothy Simon EVANS, Woodstock, Oxfordshire (GB); Edward Thomas George BARRETT, Woodstock, Oxfordshire (GB)

(21) Appl. No.: 18/263,921

(22) PCT Filed: Feb. 3, 2022
(86) PCT No.: PCT/EP2022/052566

§ 371 Date: Aug. 2, 2023

(30) Foreign Application Priority Data
Feb. 4, 2021 (GB) 2101584.7
Feb. 4, 2021 (GB) 2101585.4
Feb. 4, 2021 (GB) 2101587.0

(51) Int. Cl. A61M 5/20

(52) U.S. Cl. CPC A61M 5/2033; A61M 2005/206

(57) ABSTRACT

Aspects of the present invention relate to an injection device comprising a drive means, a plunger, and an intermediate member disposed between the drive means and the plunger and releasably engaged with the plunger thereby to selectively transmit force from the drive means to the plunger.

### TECHNICAL FIELD

The present disclosure relates to an injection device.

### BACKGROUND

One of the most common routes of administration for medications is by injection, such as intravenous, subcutaneous or intramuscular injection. A syringe containing the medication is used for the injection, which typically is carried out by trained medical personnel. In certain instances, a patient is trained in the use of the syringe to allow for self-injection. Moreover, certain medications are formulated in pre-filled syringes for patient use, to avoid the need for the patient to fill the syringe. Some patients, however, may be averse to carrying out self-injection, particularly if the patient has a fear of needles. Automatic injection devices offer an alternative to a syringe for delivering a medication.

### SUMMARY OF THE INVENTION

Aspects and embodiments of the invention provide an injection device as claimed in the appended claims.

According to an aspect of the present invention there is provided an injection device comprising a syringe carrier configured to receive a medicament container; a housing having a proximal end and a distal end; the housing being configured to receive the syringe carrier and releasably engage the proximal end of the syringe carrier; and a proximal end cap arranged to engage with the proximal end of the housing and comprising a wall extending distally along the housing, the proximal end cap configured to maintain the engagement between the proximal end of the syringe carrier and the housing. The wall acts to prevent the syringe carrier moving proximally whilst the injection device has a proximal cap on and therefore reduces the chances of actuation of the device before it is intended.

Optionally, the housing further comprises an abutment feature that the syringe carrier abuts against. The abutment feature may be a projection extending inwardly from the inner surface of the housing towards the central longitudinal axis of the injection device.

The projection may be, for example, an annular projection extending around the inner circumference of the housing, a single projection or a plurality of spaced apart projections.

Optionally, the syringe carrier may have a proximal end and a distal end and the proximal end of the syringe carrier comprises a flexible finger; the proximal end of the flexible finger engaging with the housing.

The wall preferably extends distally and internally within the syringe carrier.

Preferably, the external surface of the wall contacts and extends past the internal surface of the abutment feature.

Preferably, the wall further comprises at least one projection extending internally towards the central longitudinal axis. This projection can be arranged to engage with a rigid needle shield covering a needle of the syringe and thus facilitate removal of the rigid needle shield together with the proximal end cap.

Preferably, the proximal end cap is provided with a radial wall situated at the proximal end of the wall. The proximal end cap is preferably further provided with a second wall extending distally from the radial wall and spaced apart from the wall. The second wall facilitates removal of the cap from the device.

Preferably, the second wall is arranged to removeably engage with the outer surface of the housing, optionally by frictional engagement with the outer surface of the housing.

According to another aspect of the present invention there is provided an injection device including a drive means; a plunger; and an intermediate member disposed between the drive means and the plunger and releasably engaged with the plunger thereby to selectively transmit force from the drive means to the plunger. The selective transmission of force from the drive means to the plunger facilitates the use of the drive means for multiple functions.

Preferably the intermediate driver has a compressed configuration and a relaxed configuration wherein in the compressed configuration the intermediate member is in engagement with the plunger to transmit force from the drive means to the plunger and in the relaxed configuration the intermediate member is released from engagement with the plunger such that force is not transmitted from the drive means to the plunger.

Preferably the injection device also includes a housing arranged to selective maintain the intermediate driver in the compressed configuration. This may be achieved, by the housing having a distal length with a reduced inner diameter to a proximal portion of the housing, the reduced inner diameter maintaining the compressed configuration of the intermediate driver. This may be achieved, for example, by one or more support ribs or an increased thickness of the wall of the housing.

The intermediate member may have an outer portion and an inner portion connected by a connecting portion. The outer portion may be made from one or more flexible fingers or a wall which is non contiguous at least at its proximal end. The flexible fingers or non-contiguous wall portion or a portion thereof are preferably closer together in the compressed configuration than the relaxed configuration.

The intermediate member is preferably provided with an angled surface which contacts a corresponding angled surface of the plunger. This acts to facilitate disengagement of the plunger and the intermediate member.

Preferably the intermediate member comprises an outer portion and an inner portion connected by a connecting portion; the outer and inner portion extending distally from the connecting portion to receive the proximal end of the drive means. This provides a seat which accurately locates the drive means relative to the intermediate member.

Preferably the drive means is a spring. Even more preferably the spring is a compression spring.

According to another aspect of the present invention there is provided an injection device including drive means, an intermediate member arranged to receive the drive means and a shroud; wherein the intermediate member and shroud are brought into engagement by the drive means. This allows the shroud to be deployed using the drive means negating the need for separate drive means to move the shroud and the plunger.

The shroud and intermediate member may be provided with complementary engagement features to aid engagement of the shroud and intermediate member. The engagement features may be, for example, a projection on one of the intermediate member and the shroud and a complementary recess on the other of the intermediate member and the shroud. Optionally, the projection may have a frusto-conical shape.

The shroud may include a distally extending arm and the engagement feature is present on a distal end of the distally extending arm. The intermediate member may provided with a proximally extending arm arranged to contact the shroud. Optionally the engagement feature may be provided on the proximal end of the proximally extending arm.

Preferably the injection device includes a housing and at least part of the distally extending arm and/or proximally extending arm is provided with an expanded outer section configured to be received in a complementary groove in the housing.

Optionally the distally extending arm is provided with a laterally extending flexible arm configured to engage with a portion of the housing to prevent distal movement of the shroud when the shroud is in its extended position.

Preferably the shroud comprises a contiguous wall at its proximal end.

The skilled person will understand that any one of the above features described above may be incorporated into any of the other aspects of the invention either separately or together.

### BRIEF DESCRIPTION OF THE DRAWINGS

One or more embodiments of the invention will now be described, by way of example only, with reference to the accompanying drawings, in which:

FIG. 1 shows a cross section through an injection device according to the present invention;

FIGS. 2a and 2b illustrate the proximal end of the injection device with the proximal end cap engaged and removed respectively;

FIGS. 3a and 3b show a cross section through the injection device with the intermediate driver in a compressed configuration

FIGS. 4a and 4b show a cross section through the injection device with the intermediate driver and the shroud being brought into engagement

FIG. 5 illustrates the intermediate driver and the shroud.

### DETAILED DESCRIPTION

The present invention relates to an injection device, components thereof, and methods for injecting a substance, such as a liquid drug or vaccine, into a patient to manage or cure a medical condition. In one embodiment, the automatic injection device is a pen, i.e., an autoinjector pen or autoinjection pen (used interchangeably herein).

An injection device according to an embodiment of the present invention is described herein with reference to Figure X.

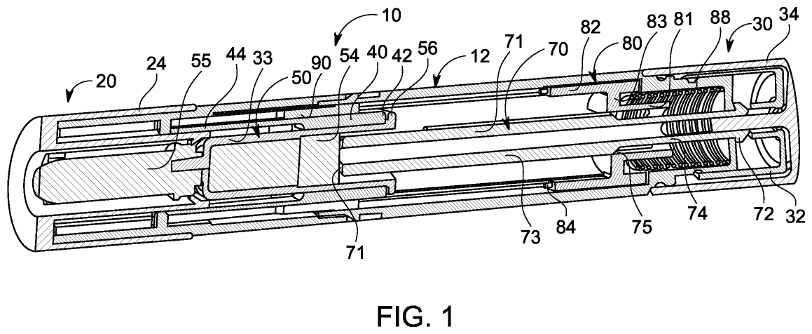

With reference to FIG. 1, the injection device 10 includes a housing 12 for housing a container, such as a syringe, containing a dose of a substance to be injected. The housing 12 preferably has a tubular configuration, though the housing 12 may have any suitable size, shape and configuration for housing a syringe or other container of a substance to be injected. While the invention will be described with respect to a syringe mounted in the housing 12, one skilled in the art will recognize that the automatic injection device 10 may employ any suitable container for storing and dispensing a substance. References to radial and axial in this specification should be taken in relation to the longitudinal axis unless otherwise stated. The term axis can be taken to be the longitudinal axis of the device unless otherwise stated or obvious from the context. The normal of the longitudinal axis is taken to be perpendicular to the longitudinal axis.

The syringe is preferably slidably mounted in the housing 12, as described in detail below. In an inactivated position, the syringe is sheathed and retracted within the housing 12. When the device is actuated, a needle of the syringe projects from a first (proximal) end 20 of the housing 12 to allow ejection of a substance from the syringe into a patient. As shown, the first end of the housing 20, i.e., the proximal end, includes an opening through which the needle of the syringe projects during actuation of the device 10.

A second (distal) end 30 of the housing 12, i.e., the distal end, includes an activation button 32 for actuating the syringe to move from a sheathed position within the housing 12 to a projecting position, and subsequently to expel the substance from the needle into the patient. The housing 12 houses one or more actuators that perform the functions of moving the syringe and expelling the substance from the syringe.

A first removable cap 24 (or needle cap) for covering the first end 20 of the housing 12, to prevent exposure of the needle in the syringe prior to use. A second removable cap 34 (or actuator cap) covers the second end 30 of the housing 12 to prevent accidental actuation of the activation button 32.

A syringe 50 or other suitable container for a substance is disposed within the interior of the housing 12. The illustrative syringe 50 includes a hollow barrel portion 53 for holding a dose of a liquid substance to be injected. The illustrative barrel portion 53 is substantially cylindrical in shape, though one skilled in the art will recognize that the barrel portion 53 may have any suitable shape or configuration. A seal, illustrated as a bung 54, seals the dose within the barrel portion 53. The syringe 50 may further include a hollow needle 55 connected to and in fluid communication with the barrel portion 53, through which the dose can be ejected by applying pressure to the bung 54. The hollow needle 55 extends from a proximal end of the barrel portion 53. The distal end of the barrel portion 53 includes a flange 56. The skilled person will recognize that the invention is not limited to the illustrative embodiment of the syringe 50 and that any suitable container for containing a dose of a substance to be injected may be used in accordance with the teachings of the invention.

The syringe 50 is received within a syringe carrier 40 which is slidably disposed within the housing 12. The syringe carrier 40, at its distal end, is provided with an abutment surface arranged to engage with the flange 56 of the syringe. At its proximal end the syringe carrier is provided with one or more flexible projections 44. The syringe 50 rests in the carrier 40, and both are contained in the housing 12. The syringe carrier 400 may have any suitable configuration and size suitable for carrying or guiding the syringe 50 within the housing 12.

The injection device further includes an actuation mechanism, including a plunger 70, an intermediate driver 80 and biasing means. In the present invention the biasing means is a compression spring 88, although it will be understood that any suitable means for biasing the plunger and intermediate driver 80 distally.

The actuation mechanism selectively moves and actuates the syringe 50 to inject the dose contained in the syringe 50 into a user. The illustrative plunger 70 includes a rod portion 71 having a first end 71a integral with, connected to or in communication with the bung 54 for selectively applying pressure to the bung 54 to expel the dose from the needle 55. The plunger 70 may include a flanged second end 72.

The rod portion 71 is provided with a proximal portion 73 and a distal portion 74 having differing external diameters and connected by an angled portion 75. In the present embodiment of the invention the external diameter of the proximal portion 73 is greater than the distal portion 74 of the rod portion 71.

The intermediate driver 80 is provided with a cylindrical inner portion 81 and an outer portion 82 connected by a connecting portion 83. The circumference of the outer portion 81 is greater than that of the inner portion 81 and the outer portion 81 extends further distally than the inner portion. The connecting portion 83 and the compression spring 88 are configured such that, prior to use of the injection device, 10, the compression spring is engaged with the connecting portion 83 and biases the intermediate driver 80 distally. Additionally, prior to use the connecting portion 83 is situated such that it is in communication with the angled portion 75 of the plunger 70 such that any biasing force exerted on the intermediate driver 80 by the compression spring 88 is communicated to the plunger 70 and distally biases the plunger 70.

The activation button of the present embodiment retains the plunger 70 and first biasing means 88 in a retracted, latched position. In the illustrative embodiment, the flanged second end 72 of the plunger 70 extends through an opening in the distal end of the housing 12 and the flanges abut the external distal wall of the housing. The flanges are configured such that proximal pressure on the flanges causes the flanged end of the plunger to flex inwards and move through the opening in the distal end of the housing 12 thereby allowing the coil spring 72 to move the intermediate driver 80 and plunger distally. For example, the second flanged end 72 of the plunger 70 may comprise one or more proximally extending fingers provided with one or more flanges.

Any suitable alternative method of releasably retaining the distal end of the plunger 70 distally may be used. The activation means which may have any suitable size, shape, configuration and location suitable for releasing the plunger 70 or otherwise activating the device 10. The activation means may be an activation button 32 formed on a distal end 30 of the housing 12, or may comprise another suitable device, such as a latch, twist-activated switch and other devices known in the art. While the illustrative activation means is located towards a distal end 30 of the device 10, one skilled in the art will recognize that the activation means may be positioned in any suitable location on the device 10.

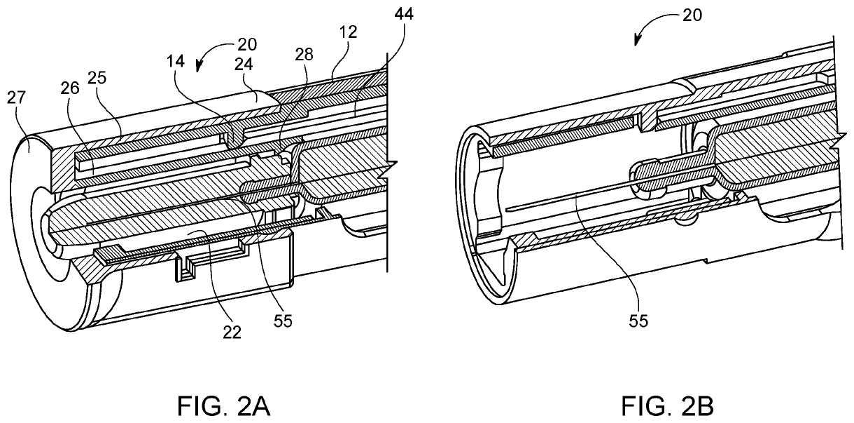

FIGS. 2a and 2b illustrate the proximal end 20 of the injection device 10 before and after the first removeable cap 24 is removed from the device. In FIG. 2a the first removeable cap 24 is engaged with the proximal end 20 of the injection device. An interior needle cover 22 sheaths the syringe needle 55.

The housing 12 is provided with at least one radial projection or abutment feature 14 which extends inwards towards the central longitudinal axis of the injection device 10. The proximal end the syringe carrier is provided with one or more flexible projections 44 which engage the at least one radial projection 14 before the injection device 10 is activated. The engagement of the at least one radial projection 14 and the proximal ends of the one or more flexible projections 44 acts to retain the syringe carrier in its distal, pre-activation, position.

The first removeable cap 24 is provided with an inner wall 25 and an outer wall 26 having a greater diameter than the inner wall 25. The inner and outer walls 25, 26 extend distally and are connected by a proximal end wall 27. The inner surface of the outer wall 26 is configured to engage with the outer surface of the proximal end of the housing 12 in any suitable manner. For example, the outer wall 25 may form a frictional engagement with the housing or be provided with a screw thread.

The inner wall 25 extends distally inside the proximal end of the housing and the proximal end of the syringe carrier. The outer surface of the inner wall 25 and the radial projection 14 are in contact when the first removeable cap 24 and housing 12 are engaged. The inner wall 25 is of sufficient rigidity to prevent the flexible projections of the syringe carrier flexing towards the central longitudinal axis of the injection device 10 and moving past the radial projection 14 when the first removeable cap 24 is engaged with the housing 12. This assists prevention of premature activation of the device by retaining the syringe carrier 24 in its distal pre-activation position.

The inner wall 25 may be provided with one or more projections 28 which extend inwardly towards the central longitudinal axis of the injection device 10. The projection(s) 28 are configured to engage with the a feature of the interior needle cover 22, for example, the distal end of the interior needle cover 22, such that removal of the first removeable cap 24 also causes removal of the interior needle cover 22 as shown in FIG. 2b. When the cap 24 is removed, the syringe needle 55 is exposed within the lumen 1012 of the housing 12. The cap 24 may also include an opening in a proximal end thereof.

Although the first removeable cap 24 has been described as having tubular walls the skilled person will understand that the cap may be any suitable shape to engage with the housing 12. Additionally the inner wall 25 may comprise one or more sections spatially separated. Further, although the first removeable cap 24 has been described as having an inner wall and an outer wall the skilled person will understand that the outer wall is optional.

To actuate the device the second removeable cap 34 is removed from the device. The injection device 10 can then be introduced to the injection site. Depressing the button 32 causes the flanged end of the plunger 70 to move inwards thereby causing the flanges to no longer be retained by distal wall of the housing 12. This allows the spring 88, which has been retained in a compression state, to exert a proximal force on the intermediate driver 80.

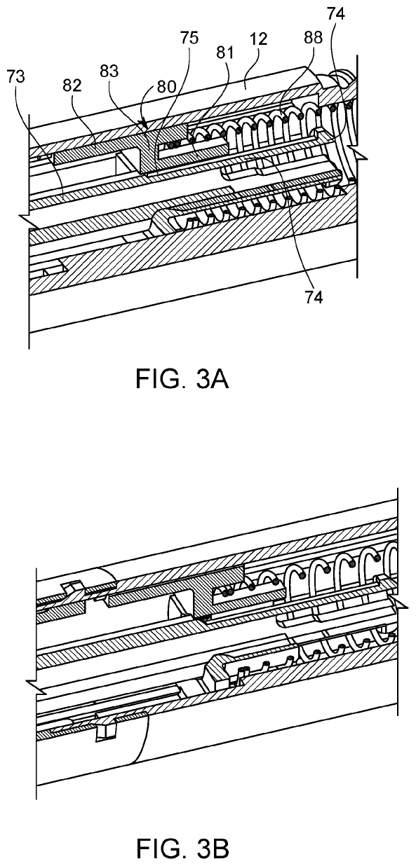

FIG. 3 illustrates the interaction between the plunger rod 70 and the intermediate driver 80. As can be seen the connecting portion 83 is provided with an inner angled surface on its proximal side which engages with the angled portion 74 of the plunger. The proximal end of the compression spring 88 is seated on the distal side of the flange and is received between the inner portion 81 and a part of the outer portion 82 which extends distally from the flange forming a seat for the proximal end of the compression spring 88.

As the compression spring 88 exerts a force on the distal side of the connecting portion 83 this force is transmitted through to the angled face of the connecting portion 83 which engages the angled portion 74 of the plunger rod 70 thereby moving the plunger rod 74 proximally. The force exerted on plunger 70 causes a force to be exerted on the syringe 50 via the bung 54 causing the flexible projections 44 of the syringe carrier 40 to be deflected inwardly and move past the abutment feature 14 and thus move proximally inserting the needle into the user.

Once the needle 55 has been inserted forward motion of the syringe carrier 40 and thus the syringe 50 is arrested by any suitable means meaning that further proximal movement of the plunger rod causes proximal movement of the bung 54 within the syringe 50 thereby causing medicament to be expelled from the container 18 through the needle 22.

In FIG. 3a the intermediate driver 80 is held in a compressed formation by the housing 12. At least the outer portion is able to move from a compressed formation with a reduced diameter to a relaxed formation with an increased internal diameter. This may be achieved, for example, by providing one or more flexible fingers as the outer portion 82, at least part of the outer portion 82 being formed by a non-contiguous wall or by making the outer portion from at least two materials, one being more flexible than the other to allow the circumference of the outer portion to be reduced.

In the compressed formation the intermediate driver 80 has an inner circumference less than the outer circumference of the proximal portion 73 of the plunger rod 70 and thus engages the plunger rod 70 at the angled portion 75. In the relaxed formation the intermediate driver 80 has an inner circumference greater than the outer circumference of the proximal portion 73 of the plunger rod 70 and thus can move along the plunger rod.

The housing is provided with one or more supporting ribs (not shown) extending longitudinally down the inner surface of the housing. The ribs reduce the inner diameter of the housing thereby maintaining the intermediate driver 80 in the compressed configuration.

Any other suitable means may be used to reduce the inner diameter of the housing. For example, the thickness of the housing wall may be increased or annular projections or rings may be provided if the longitudinal distance between the annular projections is less than the length of the flexible part of the outer portion.

The inner diameter of the housing increases at a position where the intermediate driver 80 is to move to its relaxed formation and thus disengage from the plunger. This may be achieved by reaching the end of the inner supporting ribs, decreasing the thickness of the housing wall, ending the annular projections. In the present device the intermediate driver 80 disengages from the plunger 70 when the medicament has been delivered to the user i.e. when the bung 54 has reached the proximal end of the syringe 50. The skilled person will understand that any suitable position may be chosen.

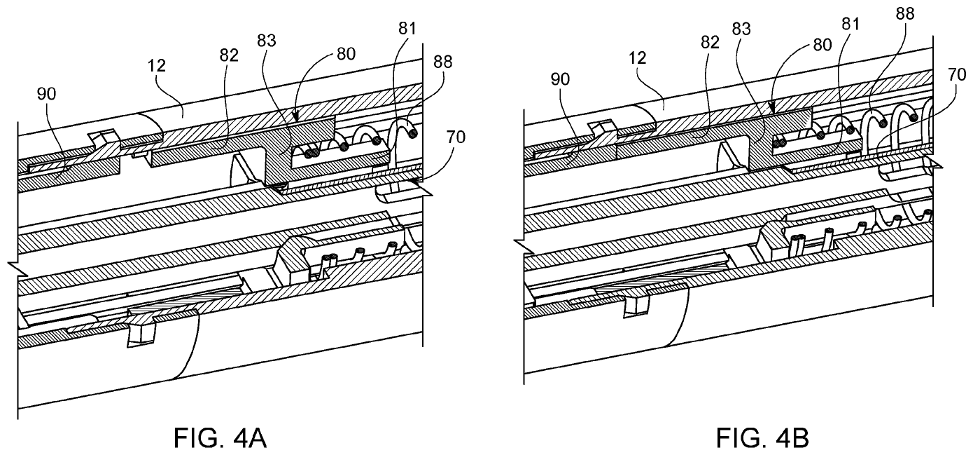

FIG. 3b shows the intermediate driver 80 reaching, for example, the end of the supporting ribs. When this occurs the outer portion can move outwards towards the relaxed position as illustrated in FIG. 4b. The outer movement of the outer portion allows the angled surfaces of the plunger and the connecting portion to move past eachother thereby disengaging the plunger 70 and intermediate driver 80. Thus the plunger 70 no longer has a proximal force being applied to it by the drive spring 88 and no longer moves forward. Additionally, drive spring 88 is still exerting a proximal force on the connecting portion of the intermediate driver 80 and thus the intermediate driver 80 continues to move proximally within the housing 12 of the injection device 10.

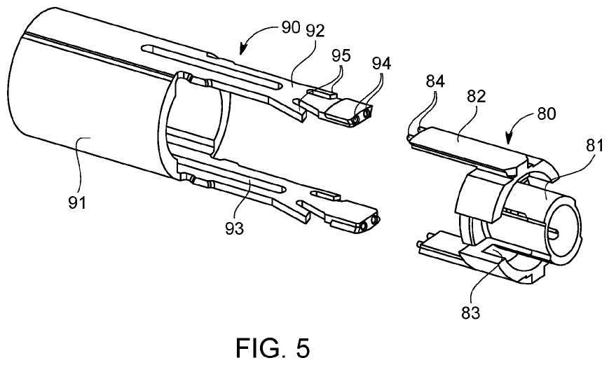

As the intermediate driver 80 continues to move proximally it contacts a shroud 90 as illustrated in FIGS. 4a and 4b. As the intermediate driver is driven proximally by the drive spring 88 it also drives the shroud 90 proximally. The shroud and intermediate driver 80 are illustrated in more detail in illustrated FIG. 5.

In FIG. 5 it can be seen that the shroud 90 is provided with a shrouding portion 91 which has a contiguous wall to protect the user from the needle after the injection device has been used. The shroud is further provided with two rearwardly extending arms 92 configured to engage with the intermediate driver. Each of the two rearwardly extending arms 92 are provided with a recess 93 which receives the radial projection 14 or abutment feature and allows the shroud 90 to move proximally along the radial projection. The radial projection 14 may guide the direction of movement of the shroud 90; however, the skilled person would also understand that the arms 92 may be positioned in order that they do not engage with the radial projection 14.

The distal end of the shroud arms 92 and the proximal end of the outer portion 82 of the intermediate driver 80 are provided with engagement features 84, 94. The engagement features may be any suitable features designed to assist the engagement of the intermediate driver 80 and the shroud 90. For example, one of the intermediate driver 80 and the shroud 90 may be provided with a projection 84 and the other of the intermediate driver 80 and the shroud 90 is provided with a complementary recess 94 to receive the projection. As illustrated in FIG. 5 the projections have a frusto-conical shape, however, the skilled person will understand any suitable shape may be used.

Additionally, in order to assist alignment of the distal end of the shroud arms 92 and the proximal end of the outer portion 82 an outer portion of the shroud arms and/or the outer portion may be received in a groove in the housing 12.

Each of the shroud arms 92 is further provided with flexible lateral arms 95. When the shroud 90 is at its proximal position the distal end of the lateral arms 95 abuts a feature in the housing 12 (not shown) thereby preventing the shroud 90 from being moved distally after the injection device has been used. The skilled person would understand that alternative methods of preventing the shroud from moving backwards may be utilised.

Certain terminology is used in the following description for convenience only and is not limiting. The word “proximal” refers to the front, or patient, end of the device. The word “distal” refers to the rear, or syringe user, end of the device. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis. It should be understood that the detailed description and specific examples are given by way of illustration only, since various changes and modifications within the spirit and scope of the disclosure will become apparent to those skilled in the art from the description.

1. An injection device comprising:
i) a drive means;
ii) a plunger;
iii) a shroud; and
iv) an intermediate member disposed between the drive means and the plunger and releasably engaged with the plunger thereby to selectively transmit force from the drive means to the plunger and from the drive means to the shroud.

2. An injection device as claimed in claim 1 wherein the intermediate driver has a compressed configuration and a relaxed configuration wherein in the compressed configuration the intermediate member is in engagement with the plunger to transmit force from the drive means to the plunger and in the relaxed configuration the intermediate member is released from engagement with the plunger such that force is not transmitted from the drive means to the plunger.

3. An injection device as claimed in claim 2 wherein the injection device further comprises a housing arranged to selective maintain the intermediate driver in the compressed configuration.

4. An injection device as claimed in claim 3 wherein the housing comprises a distal length with a reduced inner diameter to a proximal portion of the housing, the reduced inner diameter maintaining the compressed configuration of the intermediate driver.

5. An injection device as claimed in claim 4 wherein the reduced diameter is provided by one or more support ribs or an increased thickness of the wall of the housing.

6. An injection device as claimed in claim 1 wherein the intermediate member comprises an outer portion and an inner portion connected by a connecting portion.

7. An injection device as claimed in claim 6 wherein the outer portion comprises one or more flexible fingers or a wall which is non contiguous at its proximal end.

8. An injection device as claimed in claim 7 wherein the flexible fingers or non-contiguous wall portion are closer together in the compressed configuration than the relaxed configuration.

9. An injection device as claimed in claim 1 wherein the intermediate member is provided with an angled surface which contacts a corresponding angled surface of the plunger.

10. An injection device as claimed in claim 1 wherein the intermediate member comprises an outer portion and an inner portion connected by a connecting portion; the outer and inner portion extending distally from the connecting portion to receive the proximal end of the drive means.

11. An injection device as claimed in claim 1 wherein the drive means is a spring.

12. An injection device as claimed in claim 11 wherein the spring is a compression spring.
